(19) United States
(12) Patent Application Publication
(10) Pub. No.: US 2024/0285674 A1
(43) Pub. Date: Aug. 29, 2024

(54) USE OF SULFATE GLUCO-GALACTO-OLIGOSACCHARIDE IN PREPARATION OF ANTICOAGULANT AND/OR ANTITHROMBOTIC DRUG

(71) Applicant: NANJING UNIVERSITY OF SCIENCE AND TECHNOLOGY, Nanjing, Jiangsu (CN)

(72) Inventors: Jianfa ZHANG, Nanjing, Jiangsu (CN); Qian LIN, Nanjing, Jiangsu (CN); Lei WANG, Nanjing, Jiangsu (CN)

(21) Appl. No.: 18/577,953

(22) PCT Filed: Jul. 4, 2022
(86) PCT No.: PCT/CN2022/103538

§ 371 Date: Jan. 9, 2024

(30) Foreign Application Priority Data
Jul. 9, 2021 (CN) 202110779428.0
Nov. 4, 2021 (CN) 202111299660.0

(51) Int. Cl. A61K 31/737; A61P 7/02; C08B 37/00

(52) U.S. Cl. CPC A61K 31/737; A61P 7/02; C08B 37/006

(57) ABSTRACT

The present invention relates to the field of anticoagulant and antithrombotic drugs, and in particular to an application of sulfate gluco-galacto-oligosaccharide in preparation of an anticoagulant and/or antithrombotic drug. Disclosed in the present invention is the application of sulfate gluco-galacto-oligosaccharide in the preparation of the anticoagulant and/or antithrombotic drug. The structural formula of sulfate gluco-galacto-oligosaccharide is that R₁-R₂₄ are independently SO₃⁻ or H, but not all H. Sulfate gluco-galacto-oligosaccharide of the present invention has obvious anticoagulant activity in vivo and in vitro, and can be used for preparing the anticoagulant and/or antithrombotic drug.

### CROSS REFERENCE TO RELATED APPLICATION

The present disclosure is a national stage application of International Patent Application No. PCT/CN2022/103538, filed on Jul. 4, 2022, which claims priority to the Chinese Patent Application CN202110779428.0 filed with the China National Intellectual Property Administration (CNIPA) on Jul. 9, 2021 and entitled “USE OF SULFATE GLUCO-GALACTO-OLIGOSACCHARIDE IN PREPARATION OF ANTICOAGULANT AND/OR ANTITHROMBOTIC DRUG”, as well as the Chinese Patent Application CN202111299660.0 filed to the China National Intellectual Property Administration (CNIPA) on Nov. 4, 2021 and entitled “USE OF SULFATE GLUCO-GALACTO-OLIGOSACCHARIDE IN PREPARATION OF ANTICOAGULANT AND/OR ANTITHROMBOTIC DRUG.” All of the above-mentioned applications are herein incorporated by reference in their entireties.

### TECHNICAL FIELD

The present disclosure relates to the technical field of anticoagulants and antithrombotic drugs, and specifically relates to use of sulfate gluco-galacto-oligosaccharide in preparation of an anticoagulant and/or antithrombotic drug.

### BACKGROUND

Blood coagulation refers to a process in which blood changes from a flowing liquid into an immobile gel, and is essentially a process in which water-soluble fibrinogen transforms into water-insoluble solid fibrin. This process includes the generation of a prothrombin activator under the intrinsic or/and extrinsic coagulation pathway, the generation of thrombin under the action of a coagulation factor, and the conversion of fibrinogen into fibrin under the action of the thrombin.

Thrombus is a small piece of blood formed on a surface of the inner surface of the blood vessel of the cardiovascular system where it is peeled off or repaired. Thrombus formation involves local blood coagulation in the vasculature and generally leads to serious illnesses such as heart attacks and strokes. Risk factors for thrombosis include abnormal hyperlipidemia, hyperglycemia, and elevated plasma fibrinogen.

Heparin is the most widely used anticoagulant drug that has been in clinical use for more than 90 years. The heparin is a mucopolysaccharide extracted from the intestinal mucosa or liver of pigs, sheep, cattle and other animals, and to a certain extent results in a waste of animal resources. Heparin can prevent and treat thrombosis, but can also produce side effects, such as easily causing spontaneous bleeding, manifested as various mucosal bleeding, joint bleeding, and wound bleeding. After the heparin is injected, a number of platelets in the blood decreases significantly. Long-term use of the heparin can lead to a decrease in bone mineral density, causing osteoporosis and increasing the risk of fractures. Low-molecular-weight heparin is produced by controlled depolymerization of the heparin. Compared with unfractionated heparin, the low-molecular-weight heparin shows higher availability and longer half-life, and has become the anticoagulant drug of choice for the treatment of deep vein thrombosis. However, low-molecular-weight heparin is essentially derived from animal tissues, and its anticoagulant effect is difficult to reverse, leading to the risk of bleeding due to overdose. Ultra-low-molecular-weight heparin is mainly a type of chemically synthesized heparinoid with desirable pharmacokinetic control and no contamination by viral impurities. However, these drugs are complex in chemical synthesis with an extremely high cost, and their anticoagulant effects are also not easily reversed. Therefore, there is an urgent clinical need to find novel antithrombotic drugs and anticoagulants with fewer side effects than those of the heparin.

Previous studies have reported a gluco-galacto-oligosaccharide and a preparation method thereof, where the gluco-galacto-oligosaccharide has a definite molecular weight, a low viscosity, and a desirable water solubility. Moreover, it is pointed out that the gluco-galacto-oligosaccharide, as a prebiotic, exhibits a biological activity to significantly improve the gut microbiota (Wang, L., Cheng, R., Sun, X., et al. Preparation and Gut Microbiota Modulatory Property of the Oligosaccharide Riclinoctaose, J. Agric. Food Chem. 2021, Mar. 31, 69(12): 3667-3676.). International patent WO2019090203A1 has disclosed a sulfated heparan sulfate oligosaccharide compound with an anti-inflammatory activity, but the compound does not have anticoagulant activity. The above examples indicate that oligosaccharides do not necessarily have anticoagulant and antithrombotic activities after sulfation modification.

### SUMMARY

An objective of the present disclosure is to provide a sulfate gluco-galacto-oligosaccharide.

The present disclosure further provides use of sulfate gluco-galacto-oligosaccharide in preparation of an anticoagulant and/or antithrombotic drug.

In the present disclosure, the sulfate gluco-galacto-oligosaccharide has a high activity in inhibiting the production of thrombin by coagulation factors, and can be used as an anticoagulant or antithrombotic drug to control thrombus.

In the present disclosure, the anticoagulant and/or antithrombotic drug are conventional anticoagulants and/or antithrombotic drugs. The drug may specifically be a drug for treating acute thromboembolic diseases, for treating cerebral infarction, atherosclerotic plaque formation in a carotid artery, pulmonary infarction, myocardial infarction, splenic infarction, thrombosis in a mesenteric vein, mesenteric arterial embolism, and a venous thromboembolic disease, and for treating lower extremity arteriosclerosis obliterans. Alternatively, the drug may be a drug for treating disseminated intravascular coagulation, or an in vitro anticoagulant drug, such as an anticoagulant of a blood sample specimen for in vitro experiments including blood transfusion, extracorporeal hemodialysis, and peritoneal dialysis.

In the present disclosure, the sulfate gluco-galacto-oligosaccharide has a structural formula shown in formula 1:

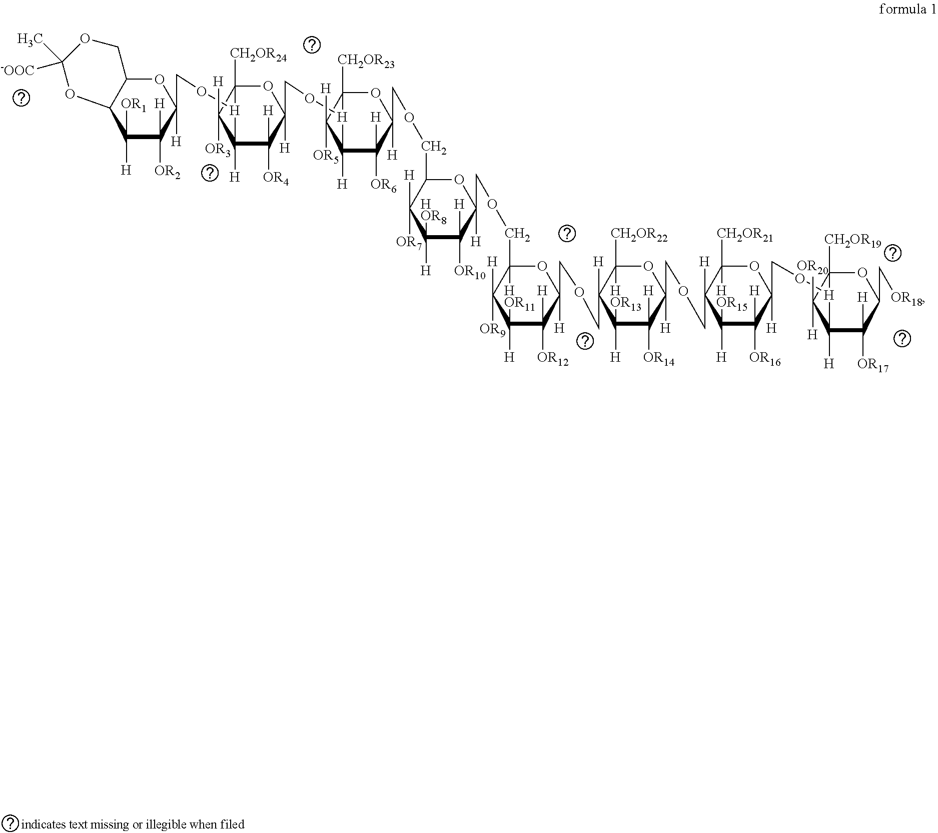

where, R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, R₂₁, R₂₂, R₂₃, and R₂₄ independently are selected from the group consisting of SO₃⁻ and H, but are not all H.

In the present disclosure, the sulfate gluco-galacto-oligosaccharide has an average of 0.5 to 3 SO₃⁻ on each sugar residue. The greater the average number of SO₃⁻ on each sugar residue, the higher the inhibition rate of FXa. In a specific example, there are preferably 0.5 to 2.5, more preferably 2.1 to 2.5 SO₃⁻ on average per sugar residue.

In the present disclosure, the sulfate gluco-galacto-oligosaccharide has 8 sugar units, including 7 glucose residues and 1 galactose residue. A 1st glucose residue is ligated to a pyruvate group, while an 8th sugar unit is a galactose residue. A specific ligation method of the 7 glucose residues (Glcp) and the 1 galactose residue (Galp) is shown in formula 2:

D-Glcp(4,6-pyr)-β-(1→3)-D-Glcp-β-(1→3)-D-Glcp-β-(1→6)-D-Glcp-β-(1→6)-D-Glcp-β-(1→4)-D-Glcp-β-(1→4)-D-Glcp-β-(1→3)-D-Galp, formula 2.

In the present disclosure, a preparation method the sulfate gluco-galacto-oligosaccharide includes: subjecting gluco-galacto-oligosaccharide to sulfation with a sulfating reagent to obtain the sulfate gluco-galacto-oligosaccharide.

In the present disclosure, a sulfation degree of the gluco-galacto-oligosaccharide can be controlled by controlling a ratio of the gluco-galacto-oligosaccharide and the sulfating reagent, a reaction temperature, and a reaction time. That is, an average number of sulfate groups on each sugar residue is controlled to obtain gluco-galacto-oligosaccharide types substituted with sulfate groups to varying degrees.

In the present disclosure, the sulfating reagent is a sulfating reagent commonly used in this field, as long as it can sulfate the gluco-galacto-oligosaccharide. In a specific example, the sulfating reagent is preferably a mixture of pyridine and chlorosulfonic acid.

In a specific example, the preparation method of the sulfate gluco-galacto-oligosaccharide specifically includes: preparing the sulfating reagent using pyridine and chlorosulfonic acid; adding a dimethyl sulfoxide (DMSO) solution of the gluco-galacto-oligosaccharide into the sulfating reagent under stirring to allow the sulfation in an oil bath at 50° C. to 120° C.; and subjecting an obtained reaction solution to a post-treatment and purification to obtain the sulfate gluco-galacto-oligosaccharide.

Preferably, the pyridine and the chlorosulfonic acid in the mixture are at a volume ratio of 3:1 to 8:1.

Preferably, the gluco-galacto-oligosaccharide and DMSO in the DMSO solution of the gluco-galacto-oligosaccharide are at a mass-to-volume ratio of 1 g:(10-20) mL.

Preferably, the gluco-galacto-oligosaccharide and the sulfating reagent are at a mass-to-volume ratio of 1 g:20 mL to 1 g:60 mL.

Preferably, the sulfation is conducted at 60° C.

Preferably, the sulfation is conducted for 2 h to 5 h.

Preferably, the post-treatment and the purification include: adjusting the reaction solution to a neutral pH value with a 15% NaOH solution, conducting centrifugation at 8,000 rpm and 4° C. to remove insoluble impurities, collecting an obtained supernatant to allow dialysis with a dialysis bag of 500 Da to 1,000 Da, subjecting a resulting dialyzate to concentration and desalination using an organic ultrafiltration membrane, and then conducting freeze-drying or ethanol precipitation to obtain the sulfate gluco-galacto-oligosaccharide.

Compared with the prior art, the present disclosure has at least the following advantages:

In the present disclosure, it is discovered for the first time that the sulfate gluco-galacto-oligosaccharide has obvious anticoagulant activity in vitro, and can selectively inhibit a coagulation factor Xa (FXa), resulting in a small risk of bleeding. Therefore, the sulfate gluco-galacto-oligosaccharide is suitable for preparing in vitro anticoagulants. At the same time, the sulfate gluco-galacto-oligosaccharide exhibits a function of inhibiting thrombosis in mouse models and can be used to control thrombosis. The sulfate gluco-galacto-oligosaccharide is safe and non-toxic, has a strong medicinal effect, and shows broad application prospects in the anticoagulant and/or antithrombotic drug.

### BRIEF DESCRIPTION OF THE DRAWINGS

FIG. 1 shows results of an inhibitory activity of the sulfate gluco-galacto-oligosaccharide against FXa, including the inhibitory activities of gluco-galacto-oligosaccharide and sulfate gluco-galacto-oligosaccharide against FXa, both at a concentration of 1 mg/mL;

FIG. 2 shows an effect of the sulfate gluco-galacto-oligosaccharide on activated partial thromboplastin time (APTT), including the effects of sulfate gluco-galacto-oligosaccharide samples of three different concentrations on plasma coagulation time;

FIG. 3 shows that the sulfate gluco-galacto-oligosaccharide inhibits the formation of arterial thrombosis induced by FeCl₃, where a group (a) is the thrombosis of mice injected with physiological saline; a group (b) is the thrombosis of mice injected with 0.5 mg/mL sulfate gluco-galacto-oligosaccharide; and a group (c) is the thrombosis of mice injected with 1.0 mg/mL sulfate gluco-galacto-oligosaccharide; and

FIG. 4 shows an effect of the sulfate gluco-galacto-oligosaccharide on a tail bleeding time of mice, including the effects of sulfate gluco-galacto-oligosaccharide samples of two different concentrations on the bleeding situation at the tail tip of mice.

### DETAILED DESCRIPTION OF THE EMBODIMENTS

Unless otherwise specified, all technical and scientific terms used in the present disclosure have the same meaning as commonly understood by one of ordinary skill in the technical field to which the present disclosure belongs. Unless otherwise specified, the reagents or materials used in the following examples can be purchased commercially or synthesized by referring to existing methods.

In the present disclosure, the gluco-galacto-oligosaccharide is prepared by conventional methods known in the art. Specifically, the gluco-galacto-oligosaccharide can be prepared according to a method taught in: Wang, L., Cheng, R., Sun, X. et al., J. Agric. Food Chem., 69, pp. 3667-3676 (2021).

In the present disclosure, the term “and/or” means use alone or in combination.

The present disclosure is described in more detail below with reference to the examples and accompanying drawings.

### Example 1

### Preparation of Sulfate Gluco-Galacto-Oligosaccharide

1.0 g of gluco-galacto-oligosaccharide was transferred to an Erlenmeyer flask, added with 10 mL to 20 mL of DMSO, and dissolved by stirring to obtain a gluco-galacto-oligosaccharide solution. Pyridine and chlorosulfonic acid were mixed in a three-necked flask at a volume ratio of 3:1, and then magnetically stirred in an ice-water bath for 30 min to obtain a sulfating reagent. The gluco-galacto-oligosaccharide solution was gradually added into 30 mL of the sulfating reagent under stirring, a condenser tube was connected while the other two bottle caps were plugged, and the three-necked flask was placed in an oil bath at 60° C. to allow a reaction by stirring for 3 h. After naturally cooling to a room temperature, the reaction was stopped. A reaction solution was adjusted to a neutral pH value with a 15% NaOH solution, centrifugation was conducted at 8,000 rpm and 4° C. to remove insoluble impurities, an obtained supernatant was collected to allow dialysis with a dialysis bag of 500 Da to 1,000 Da, a resulting dialyzate was subjected to concentration and desalination using an organic ultrafiltration membrane, and then freeze-drying or ethanol precipitation was conducted to obtain the sulfate gluco-galacto-oligosaccharide with averagely 1.65 sulfate groups per sugar residue.

According to the above preparation method, the gluco-galacto-oligosaccharide and the sulfating reagent were subjected to sulfation at a mass-to-volume ratio (g:mL) of 1:20, 1:30, 1:45, and 1:60, to obtain sulfate gluco-galacto-oligosaccharide samples with degrees of substitution (i.e., the average number of sulfate groups on each sugar residue) of 1.23, 1.65, 2.10, and 2.52, respectively.

### Determination of the Degree of Sulfation Substitution (Ds) of Sulfate Gluco-Galacto-Oligosaccharide

200 μL of 10 mg/mL sulfate gluco-galacto-oligosaccharide solution was added into a glass bottle, added with an equal volume of 4 M HCl, and complete acid hydrolysis was conducted at 100° C. for 8 h, and a resulting product was blown dry with nitrogen and then redissolved with 100 L of pure water. A 0.5% gelatin solution was prepared and added with 0.5% BaCl₂ when using. 100 μL of the sulfate gluco-galacto-oligosaccharide solution was mixed with an equal volume of BaCl₂ and the gelatin solution, and an OD value was measured at 360 nm. The Ds was calculated by the following equation 1:

$$DS = \frac{162 * S \%}{32 - 102 * S \%} , \qquad equation 1$$

The dosage of sulfating reagent, reaction time, reaction temperature and other conditions all affected the degree of sulfation substitution. The dosage of sulfating reagent was reduced, and the degree of sulfation substitution could also be reduced.

In Examples 2 to 5, unless otherwise specified, the sulfate gluco-galacto-oligosaccharide was the sulfate gluco-galacto-oligosaccharide with averagely 1.65 sulfate groups on each sugar residue.

### Example 2

### Determination of the Inhibitory Activity of Sulfate Gluco-Galacto-Oligosaccharide on Coagulation Factor FXa

### 1. Determination of Inhibitory Activity

a. 100 μL of FXa (5 nM) and 50 μL of the sulfate gluco-galacto-oligosaccharide sample (1 mg/mL) were mixed in a 96-well plate, incubated at 37° C. for 1 h, added with 50 μL of a substrate CS-11(22) (1.77 mM), and detected at a wavelength of 405 nm for 60 min, detecting once every minute.
b. 100 μL of HFXIa (human coagulation factor XIa, 1 nM) and 50 μL of the sulfate gluco-galacto-oligosaccharide sample (1 mg/mL) were mixed in a 96-well plate, incubated at 37° C. for 1 h, added with 50 μL of a substrate S2366 (1 mM), and detected at a wavelength of 405 nm for 60 min, detecting once every minute.
c. 100 μL of FXIIa (4 nM) and 50 μL of the sulfate gluco-galacto-oligosaccharide sample (1 mg/mL) were mixed in a 96-well plate, incubated 37° C. for 1 h, added with 50 μL of a substrate Pefachrome FXIIa/TH5253 (1 mM), and detected at a wavelength of 405 nm for 60 min, detecting once every minute.
d. 100 μL of Trypsin (8.3 nM) and 50 μL of the sulfate gluco-galacto-oligosaccharide sample (1 mg/mL) were mixed in a 96-well plate, incubated 37° C. for 1 h, added with 50 μL of a substrate CS-11(22) (1 mM), and detected at a wavelength of 405 nm for 60 min, detecting once every minute.
e. 100 μL of Thrombin (0.2 IU/mL) and 50 μL of the sulfate gluco-galacto-oligosaccharide sample (1 mg/mL) were mixed in a 96-well plate, incubated at 37° C. for 1 h, added with 50 μL of a substrate CS-01(38) (400 μM), and detected at a wavelength of 405 nm for 60 min, detecting once every minute.
f. 100 μL of Kallikrein (1 nM) and 50 μL of the sulfate gluco-galacto-oligosaccharide sample (1 mg/mL) were mixed in a 96-well plate, incubated 37° C. for 1 h, added with 50 μL of a substrate CS-31(02) (1 mM), and detected at a wavelength of 405 nm for 60 min, detecting once every minute.

### 2. Data Analysis

Data processing was conducted using the software Graphpad Prism 6.0. Regression analysis was conducted with the reaction time as an abscissa and the absorbance value at 405 nm as an ordinate.

The inhibitory activity of sulfate gluco-galacto-oligosaccharide against other enzymes was measured using a chromogenic substrate method to determine whether the sulfate gluco-galacto-oligosaccharide had a selective specificity for FXa. As shown in Table 1, the sulfate gluco-galacto-oligosaccharide had the selective specificity for FXa but had no inhibitory activity against other coagulation factors.

TABLE 1
Inhibitory effects of sulfate gluco-galacto-oligosaccharide
on different coagulation factors

| Coagulation factor | Inhibitory effect |
|---|---|
| Coagulation factor FXa | Inhibition |
| Coagulation factor FXIa | No inhibition |
| Coagulation factor FXIIa | No inhibition |
| Trypsin | No inhibition |
| Thrombin | No inhibition |
| Kallikrein | No inhibition |

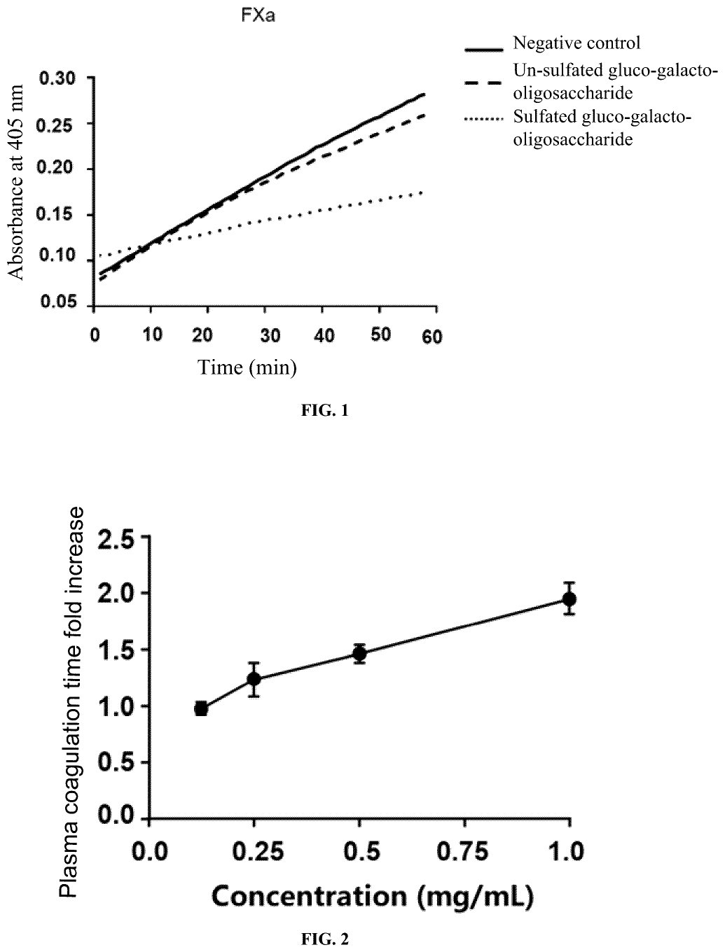

An absorbance value-reaction time curve was plotted with data points generated by a microplate reader, where the slope was a speed V of the enzymatic reaction, the negative control group had a reaction speed V0, and the sample had a reaction speed Vi. The calculated inhibition rate=(V0−V1)/V0×100%. According to FIG. 1, when sulfate gluco-galacto-oligosaccharide with averagely 1.65 sulfate groups per sugar residue was at 1 mg/mL, the inhibition rate of FXa was 57.6%. This indicated that the sulfate gluco-galacto-oligosaccharide had the inhibitory activity on FXa, while the gluco-galacto-oligosaccharide had no inhibitory activity on FXa. The degree of sulfation of the sulfate gluco-galacto-oligosaccharide was controlled by controlling the ratio of gluco-galacto-oligosaccharide and sulfating reagent. Table 2 showed the inhibitory activity of sulfate gluco-galacto-oligosaccharide samples with different sulfation degrees on FXa. It was seen that the more sulfate groups on average per sugar residue, the higher the inhibition rate of FXa by the sulfate gluco-galacto-oligosaccharide.

TABLE 2
Inhibition rate of FXa by sulfate gluco-galacto-oligosaccharide
samples with different sulfation degrees

| Gluco-galacto- | | |
|---|---|---|
| oligosaccharide:sulfating | Average number of sulfate | Inhibition |
| reagent (m:v, g:mL) | groups per sugar residue | rate |
| 1:10 | 0.47 | 10.2% |
| 1:20 | 1.23 | 39.2% |
| 1:30 | 1.65 | 57.6% |
| 1:45 | 2.10 | 65.5% |
| 1:60 | 2.52 | 78.4% |

### Example 3

### Effect of Sulfate Gluco-Galacto-Oligosaccharide on Coagulation Time

### 1. Establishment of a Mouse Model:

The experimental animals were male C57/BL-6J mice, weighing 18 g to 20 g. The mice were housed under standard experimental conditions with a 12-hour light-12-hour dark cycle with free access to water and food. The mice were anesthetized by intraperitoneal injection of 5% chloral hydrate, and administration of the drug was administered through the tail vein after confirming that the mice were completely anesthetized and had no eyelid reflex. The mice were randomly divided into four groups. A first group was a negative control group and was injected with physiological saline through the tail vein; a second group was a low-dose group, with 0.25 mg/mL sulfate gluco-galacto-oligosaccharide injected into the tail vein; a third group was a medium-dose group, with 0.5 mg/mL sulfate gluco-galacto-oligosaccharide injected into the tail vein; and a fourth group was a high-dose group, with 1.0 mg/mL sulfate gluco-galacto-oligosaccharide injected into the tail vein. Blood was collected from the abdominal aorta of mice and placed in silanized tubes moistened with anticoagulant, and then incubated at 37° C.

### 2. Determination of APTT

Activated partial thromboplastin time (APTT) is a duration required to observe plasma coagulation after adding an APTT reagent and calcium ions to the tested plasma. The APTT mainly reflects the condition of the endogenous coagulation system. Whole blood was collected and diluted appropriately, centrifuged at 1,080 rpm for 10 min, and an upper layer of platelet-rich plasma was removed. After centrifugation at 3,000 rpm for 5 min, a platelet-poor plasma (PPP) in the upper layer was taken for coagulation time measurement. The APTT was measured according to the operating procedures in the kit instructions, and the fibrin formation time was read on the coagulometer.

### 3. Data Analysis

Data processing was conducted using the Graphpad Prism 6.0 software. An APTT line chart was plotted with the sample concentration as an abscissa and the plasma coagulation time multiple as an ordinate. P<0.05 indicated a significant difference.

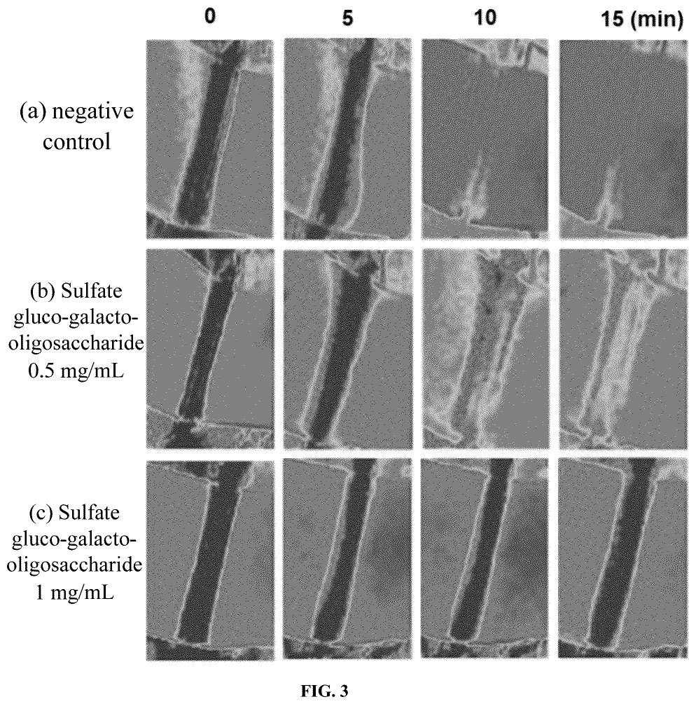

The coagulation time of the physiological saline group was recorded as 1 times, and the multiples of APTT of the sulfate gluco-galacto-oligosaccharide samples with different concentrations relative to the physiological saline group were calculated. As shown in FIG. 2, different concentrations of sulfate gluco-galacto-oligosaccharide samples had a significant impact on APTT, such that the sulfate gluco-galacto-oligosaccharide could act on the intrinsic coagulation pathway.

### Example 4

### In Vivo Antithrombotic Effects of Sulfate Gluco-Galacto-Oligosaccharide

### 1. Establishment of Animal Models:

The experimental animals were male C57/BL-6J mice, weighing 18 g to 20 g. The mice were housed under standard experimental conditions with a 12-hour light-12-hour dark cycle with free access to water and food. The mice were anesthetized by intraperitoneal injection of 5% chloral hydrate, and administration of the drug was administered through the tail vein after confirming that the mice were completely anesthetized and had no eyelid reflex. The mice were randomly divided into three groups. A first group was a negative control group and was injected with physiological saline through the tail vein; a second group was a low-dose group, with 0.5 mg/mL sulfate gluco-galacto-oligosaccharide injected into the tail vein; and a third group was a high-dose group, with 1.0 mg/mL sulfate gluco-galacto-oligosaccharide injected into the tail vein. After tail vein administration, the mouse carotid arteries were dissected, and a filter paper piece (1 mm×2 mm) dipped in 6.0% ferric chloride solution was applied to the outer wall of the mouse carotid artery to allow induction for 3 min to construct a ferric chloride-induced carotid artery thrombosis model.

### 2. Determination of Blood Vessel Occlusion Time

The blood flow and thrombosis of the mouse carotid arteries were observed using a laser speckle flow imager, and the mouse carotid arteries were observed under the moorFLPI-2 lens for 15 min. The carotid arteries of the mice at the end of the observation were carefully dissected and stained with HE.

### 3. Data Analysis

Charts were produced using GraphPad Prism 6.0 software, and statistical differences were analyzed using one-way analysis of variance and Dunnett's multiple comparisons. P<0.05 indicated a significant difference.

Blood vessel occlusion time refers to a duration from removal of the filter paper soaked in ferric chloride to the blood vessel occlusion. If no thrombosis was observed 15 min after removal of the ferric chloride filter paper, 15 min was considered as the vessel occlusion time. Combined with FIG. 3, in the negative control group (physiological saline group), the average blood vessel occlusion time was 4.5±0.23 min. Under the dosages of 0.5 mg/mL and 1.0 mg/mL of sulfate gluco-galacto-oligosaccharide in the sample, the average blood vessel occlusion times were 5.4±1.37 min and 15 min, respectively. Blood flow remained unchanged at a dose of 1.0 mg/mL, indicating that the sulfate gluco-galacto-oligosaccharide had significant antithrombotic effects.

### Example 5

Determination of Bleeding Risk with Sulfate Gluco-Galacto-Oligosaccharide

### 1. Establishment of Animal Models:

The experimental animals were male C57/BL-6J mice, weighing 18 g to 20 g. The mice were housed under standard experimental conditions with a 12-hour light-12-hour dark cycle with free access to water and food. The mice were anesthetized by intraperitoneal injection of 5% chloral hydrate, and administration of the drug was administered through the tail vein after confirming that the mice were completely anesthetized and had no eyelid reflex. The mice were randomly divided into three groups. A first group was a negative control group and was injected with physiological saline through the tail vein; a second group was a low-dose group, with 0.5 mg/mL sulfate gluco-galacto-oligosaccharide injected into the tail vein; and a third group was a high-dose group, with 1.0 mg/mL sulfate gluco-galacto-oligosaccharide injected into the tail vein.

### 2. Determination of Tail Tip Bleeding Time:

10 min after administration, the mouse was placed stably with its tail in a naturally straightened state. The mouse tail was cut off 3 mm from the tip, and then immersed in a silanized tube filled with 37° C. physiological saline. Start timing from cutting off the tail tip, 20 min. the accumulated bleeding time t after cutting off the tail tip of the mouse was recorded with a stopwatch. If the bleeding time t was greater than 20 min, it was recorded as 20 min.

### 3. Data Analysis

Data processing was conducted using the Graphpad Prism 6.0 software. A dot plot of sample concentration was made versus bleeding time. P<0.05 indicated a significant difference.

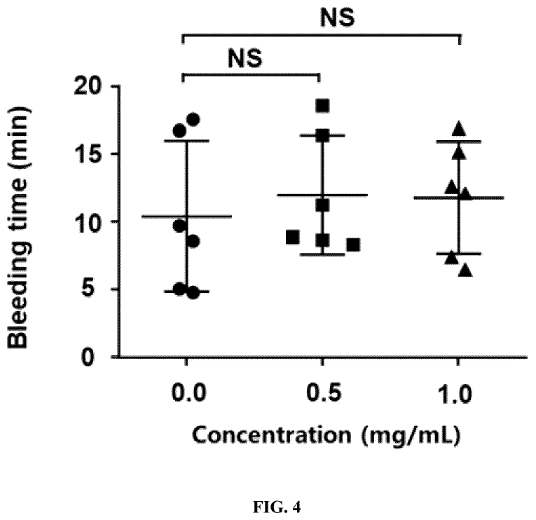

Bleeding time refers to a duration required from bleeding to natural hemostasis after capillary damage. In the negative control group (physiological saline group), the cumulative bleeding time was 10.35±5.48 min. At the dosages of 0.5 mg/mL and 1.0 mg/mL of sulfate gluco-galacto-oligosaccharide, the cumulative bleeding times were 11.88±4.44 min and 11.60±4.13 min, respectively, with no statistically significant difference (P>0.05). Combined with FIG. 4, it indicated that the sulfate gluco-galacto-oligosaccharide had a significant anti-thrombotic activity without obvious bleeding risk.

The above description is a general description of the present disclosure. Changes in form and substitutions of equivalent value may be made according to circumstances or actual needs. Although specific terminology is employed herein, these terms are intended to be descriptive and not for purposes of limitation. Various changes and modifications may be made on the present disclosure by those skilled in the prior art, and these equivalent forms also fall within the scope defined by the appended claims of the present disclosure.

1. An anticoagulant and/or antithrombotic composition, wherein the anticoagulant and/or antithrombotic composition comprises sulfate gluco-galacto-oligosaccharide, the sulfate gluco-galacto-oligosaccharide having a structural formula shown in formula 1: 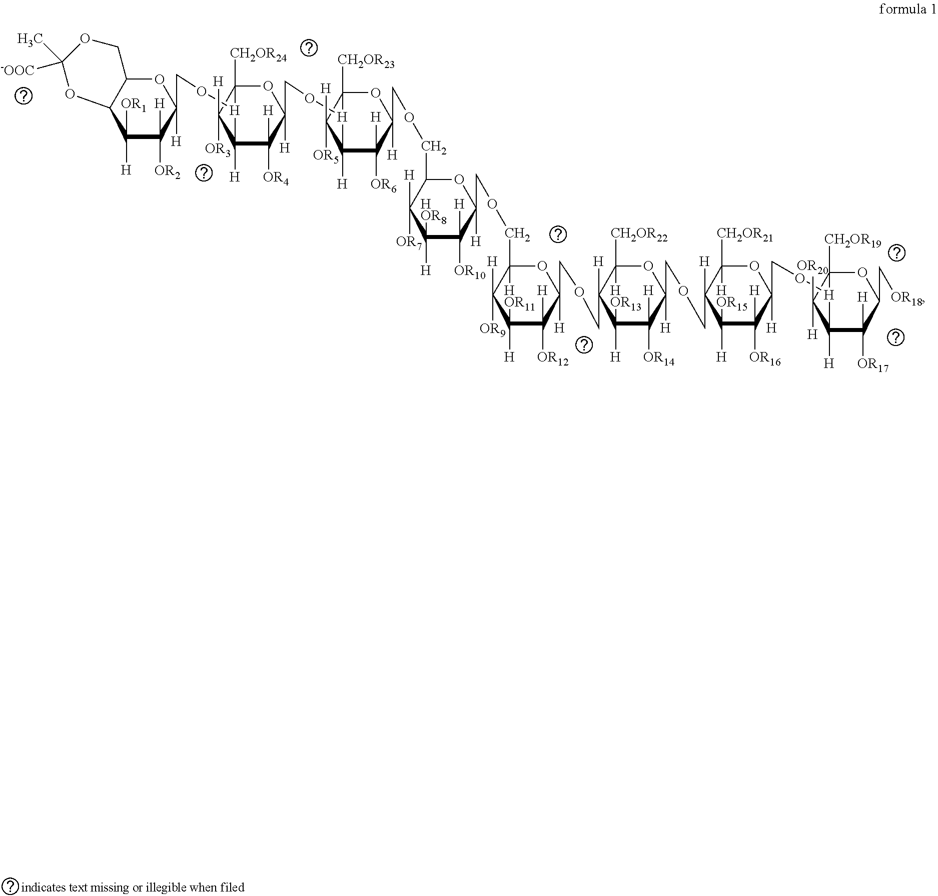
wherein, R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, R₂₁, R₂₂, R₂₃, and R₂₄ independently are selected from the group consisting of SO₃⁻ and H, but are not all H.

2. The anticoagulant and/or antithrombotic composition according to claim 1, wherein the antithrombotic composition is selected from the group consisting of a drug for treating an acute thromboembolic disease, a drug for treating cerebral infarction, atherosclerotic plaque formation in a carotid artery, pulmonary infarction, myocardial infarction, splenic infarction, thrombosis in a mesenteric vein, mesenteric arterial embolism, and a venous thromboembolic disease, and a drug for treating lower extremity arteriosclerosis obliterans; and
wherein the anticoagulant composition is selected from the group consisting of a drug for treating disseminated intravascular coagulation and an anticoagulant of a blood sample specimen for blood transfusion, extracorporeal hemodialysis, or peritoneal dialysis.

3. The anticoagulant and/or antithrombotic composition according to claim 1, wherein the sulfate gluco-galacto-oligosaccharide has an average of 0.5 to 3 SO₃⁻ on each sugar residue.

4. The anticoagulant and/or antithrombotic composition according to claim 3, wherein the sulfate gluco-galacto-oligosaccharide has an average of 0.5 to 2.5 SO₃⁻ on each sugar residue.

5. The anticoagulant and/or antithrombotic composition according to claim 4, wherein the sulfate gluco-galacto-oligosaccharide has an average of 2.1 to 2.5 SO₃⁻ on each sugar residue.

6. The anticoagulant and/or antithrombotic composition according to claim 1, wherein a preparation method of the sulfate gluco-galacto-oligosaccharide comprises: subjecting gluco-galacto-oligosaccharide to sulfation with a sulfating reagent to obtain the sulfate gluco-galacto-oligosaccharide.

7. The anticoagulant and/or antithrombotic composition use-according to claim 6, wherein the sulfating reagent is a mixture of pyridine and chlorosulfonic acid.

8. The anticoagulant and/or antithrombotic composition according to claim 6, wherein the preparation method of the sulfate gluco-galacto-oligosaccharide specifically comprises: preparing the sulfating reagent using pyridine and chlorosulfonic acid; adding a dimethyl sulfoxide (DMSO) solution of the gluco-galacto-oligosaccharide into the sulfating reagent under stirring to allow the sulfation in an oil bath at 50° C. to 120° C.; and subjecting an obtained reaction solution to a post-treatment and purification to obtain the sulfate gluco-galacto-oligosaccharide.

9. The anticoagulant and/or antithrombotic composition according to claim 8, wherein the pyridine and the chlorosulfonic acid in the mixture are at a volume ratio of 3:1 to 8:1.

10. The anticoagulant and/or antithrombotic composition according to claim 8, wherein the gluco-galacto-oligosaccharide and DMSO in the DMSO solution of the gluco-galacto-oligosaccharide are at a mass-to-volume ratio of 1 g:(10-20) mL.

11. The anticoagulant and/or antithrombotic composition according to claim 6, wherein the gluco-galacto-oligosaccharide and the sulfating reagent are at a mass-to-volume ratio of 1 g:20 mL to 1 g:60 mL.

12. The anticoagulant and/or antithrombotic composition according to claim 8, wherein the sulfation is conducted at 60° C. for 2 h to 5 h.

13. The anticoagulant and/or antithrombotic composition according to claim 8, wherein the post-treatment and the purification comprise: adjusting the reaction solution to a neutral pH value with a 15% NaOH solution, conducting centrifugation at 8,000 rpm and 4° C. to remove insoluble impurities, collecting an obtained supernatant to allow dialysis with a dialysis bag of 500 Da to 1,000 Da, subjecting a resulting dialyzate to concentration and desalination using an organic ultrafiltration membrane, and then conducting freeze-drying or ethanol precipitation to obtain the sulfate gluco-galacto-oligosaccharide.

14. The anticoagulant and/or antithrombotic composition according to claim 2, wherein the sulfate gluco-galacto-oligosaccharide has an average of 0.5 to 3 SO₃⁻ on each sugar residue.

15. The anticoagulant and/or antithrombotic composition according to claim 14, wherein the sulfate gluco-galacto-oligosaccharide has an average of 0.5 to 2.5 SO₃⁻ on each sugar residue.

16. The anticoagulant and/or antithrombotic composition according to claim 15, wherein the sulfate gluco-galacto-oligosaccharide has an average of 2.1 to 2.5 SO₃⁻ on each sugar residue.

17. The anticoagulant and/or antithrombotic composition according to claim 7, wherein the gluco-galacto-oligosaccharide and the sulfating reagent are at a mass-to-volume ratio of 1 g:20 mL to 1 g:60 mL.

18. The anticoagulant and/or antithrombotic composition according to claim 8, wherein the gluco-galacto-oligosaccharide and the sulfating reagent are at a mass-to-volume ratio of 1 g:20 mL to 1 g:60 mL.

19. The anticoagulant and/or antithrombotic composition according to claim 1, further comprising a pharmaceutical acceptable carrier.

20. A method of making the anticoagulant and/or antithrombotic composition of claim 1.
